(19) United States
(12) Patent Application Publication
(10) Pub. No.: US 2022/0186316 A1
(43) Pub. Date: Jun. 16, 2022

(54) Method and System for Microbiome-Derived Companion Diagnostics

(71) Applicant: PSOMAGEN INC., Rockville, MD (US)

(72) Inventors: Zachary Apte, San Francisco, CA (US); Jessica Richman, San Francisco, CA (US); Daniel Almonacid, San Francisco, CA (US); Rodrigo Ortiz, San Francisco, CA (US); Eduardo Morales, San Francisco, CA (US)

(73) Assignee: PSOMAGEN INC., Rockville, MD (US)

(21) Appl. No.: 17/432,183

(22) PCT Filed: Feb. 20, 2020
(86) PCT No.: PCT/US2020/019028

§ 371 Date: Aug. 19, 2021

Related U.S. Application Data
(60) Provisional application No. 62/807,760, filed on Feb. 20, 2019.
(60) Provisional application No. 62/807,761, filed on Feb. 20, 2019.
(60) Provisional application No. 62/808,304, filed on Feb. 21, 2019.

(51) Int. Cl. C12Q 1/6886; C12Q 1/689; G16B 20/20; G16B 30/00; G16B 5/00; G16B 50/00

(52) U.S. Cl. CPC C12Q 1/6886; C12Q 1/689; G16B 50/00; G16B 30/00; G16B 5/00; G16B 20/20

(57) ABSTRACT

A method for characterizing compatibility of a drug for a user includes collecting a set of samples from a set of individuals comprising a set of first individuals who respond to a therapy for a microbiome-related condition and a set of second individuals who do not respond to the therapy for the microbiome-related condition and determining one or more datasets based on the set of samples. A set of microbiome features is extracted from the one or more microbiome datasets, the microbiome features facilitating differentiation between individuals who respond and individuals who do not respond to the therapy. A companion diagnostics model is determined based on the set of microbiome features. The compatibility of the drug for the user is then determined using the companion diagnostics model.

### CROSS REFERENCE TO RELATED APPLICATIONS

This application claims the benefit of U.S. Provisional Applications 62/807,761 filed on Feb. 20, 2019; 62/808,304 filed on Feb. 21, 2019; and 62/807,760 filed on Feb. 20, 2019, in the United States Patent and Trademark Office, the disclosures of each of which are incorporated by reference herein in their entireties.

### SEQUENCE LISTING SUBMISSION VIA EFS-WEB

A computer readable text file, entitled “SequenceListing.txt,” created on Mar. 20, 2020 with a file size of 803 bytes contains the sequence listing for this application and is hereby incorporated by reference in its entirety.

### TECHNICAL FIELD

This disclosure relates generally to the field of medical diagnostics and more specifically to a new and useful method and system for microbiome-derived diagnostics and therapeutics in the field of medical diagnostics.

### BACKGROUND

The microbiome has great potential to better explain how well or badly the response to a drug will be, drug toxicity profiles, and in general how they will interact with the human body. The success or failure of clinical trials can depend on how the human microbiome interacts with the drug candidate.

### SUMMARY OF THE DISCLOSURE

In an aspect of the disclosure, a method of treating a cancer related condition associated with HPV infection includes generating a disease characterization model generated by analyzing one or more microbiome features obtained from microbiome samples from a population having the HPV-associated condition. A microbiome sample is collected from an individual. One or more microbiome datasets are generated based on the microbiome sample from the individual and a set of microbiome features is then extracted from the one or more microbiome datasets. The method further includes determining whether the individual has the HPV-associated condition based on the set of microbiome features and the disease characterization model and upon a diagnosis that the individual has the HPV-associated condition, administering a treatment based on aminolevulinic acid (ALA) in a region affected by the HPV-associated condition.

In another aspect of the disclosure, a method of treating a DNA alkylation-associated condition includes obtaining a microbiome sample from an individual. A DNA alkylation-associated condition is detected based on presence and/or amount of one of colibactin or colibactin-like compound in the microbiome sample. A colibactin-inhibiting treatment is administered to the individual for treating the DNA alkylation-associated condition.

In yet another aspect of the disclosure, a method for characterizing compatibility of a drug for a user includes collecting a set of microbiome samples from a set of individuals comprising a set of first individuals who respond to a therapy for a microbiome-related condition and a set of second individuals who do not respond to the therapy for the microbiome-related condition and determining one or more datasets based on the set of samples. A set of microbiome features is extracted from the one or more microbiome datasets, the microbiome features facilitating differentiation between individuals who respond and individuals who do not respond to the therapy. A companion diagnostics model is determined based on the set of microbiome features. The compatibility of the drug for the user is then determined using the companion diagnostics model.

Additional advantages of the present invention will become readily apparent to those skilled in this art from the following detailed description, wherein only the preferred embodiment of the invention is shown and described, simply by way of illustration of the best mode contemplated of carrying out the invention. As will be realized, the invention is capable of other and different embodiments, and its several details are capable of modifications in various obvious respects, all without departing from the invention. Accordingly, the drawings and description are to be regarded as illustrative in nature, and not as restrictive.

### BRIEF DESCRIPTION OF DRAWINGS

FIG. 1 shows a matrix of 32 bacterial targets and 19 HPV targets covered by a women's health assay and their associated health conditions.

FIG. 2A shows healthy ranges for bacterial targets for various genus of bacteria in an assay according to an embodiment of the present disclosure.

FIG. 2B shows ranges for bacterial targets for various species of bacteria in an assay according to an embodiment of the present disclosure

FIG. 3A shows a comparison of hrHPV amplification and genotyping to the digene HC2 HPV assay in accordance with an embodiment of the present disclosure.

FIG. 3B shows a comparison of lrHPV amplification and genotyping to the digene HC2 HPV assay in accordance with an embodiment of the present disclosure.

FIG. 4A shows a distribution of hrHPV types in one of the studies.

FIG. 4B shows a distribution of lrHPV types in one of the studies.

### DETAILED DESCRIPTION

The following description of the technology of the present disclosure is not intended to limit the technology to any particular embodiment, but rather to enable any person skilled in the art to make and use the technology.

Embodiments of the system and/or method can include, provide, and/or use one or more microbiome tests (e.g., kits, test components, assays, etc.) that can output, indicate, be associated with, be used for, and/or can otherwise facilitate companion diagnostics and/or determine one or more companion diagnostic-related metrics, such as for describing for one or more users (e.g., indicating to one or more users) whether and/or to what degree one or more drugs are compatible with the user. In a specific example, the method and/or system can indicate whether a drug is compatible with the user. In a specific example, the method and/or system can be implemented to indicate whether one or more users should take a drug (and/or type of drug, dosage, risk factors, pharmacogenetics, response, side effects, toxicity, characteristics of taking the drug, etc.) or not (and/or risk factors, reasoning, etc.).

Embodiments of the method and/or system can facilitate characterization of drugs (e.g., drugs going through trials and/or validation) such as by segmenting the patient population to determine which drugs work with which patients based on their microbiome.

Embodiments of the method and/or system can include and/or use companion diagnostics based on the microbiome for evaluating toxicity and/or adverse effects a drug may cause in a subset of the population carrying specific risk factors. Embodiments can include companion diagnostics and/or generating companion diagnostics (e.g., kits, tests, etc.). In examples, embodiments (e.g., kits, tests, generation of kits and/or tests, etc.) can incorporate microbiome information independent of and/or in combination with host genome information.

Embodiments can include: pairing a microbiome based test to determine if a therapeutic should be used for a given patient or not.

In a variation, the method can include evaluating and/or validating a specific set of microorganisms in relation to their effect and/or contribution for a drug therapy, such as for using that information for improving a current therapy and/or developing new therapies.

Embodiments of the method can include: collecting a set of samples from a set of individuals comprising a set of first individuals who respond (e.g., positively; etc.) to one or more therapies (e.g., for one or more microbiome-related conditions; for any suitable conditions; etc.), and a set of second individuals who do not respond (e.g., lack a positive response; etc.) to the one or more therapies; determining one or more microbiome datasets based on the set of samples, wherein the microbiome datasets comprise at least one of: a microbiome taxonomic composition dataset, a microbiome function dataset, a microbiome composition diversity dataset, and a microbiome functional diversity dataset; extracting a set of microbiome features from the at least one dataset, wherein the set of microbiome features facilitate differentiation between individuals who respond and individuals who do not respond to the one or more therapies; determining a companion diagnostics model (e.g., classifier) based on the set of microbiome features; and/or generating and/or performing companion diagnostics using the companion diagnostics model, such as for differentiating responders from non-responders in relation to the one or more therapies. However, any suitable individuals can be used for samples; any suitable microbiome data can be used for determining features; any suitable type of model can be generated for facilitating companion diagnostics; and companion diagnostics models can be used for any suitable purpose.

Embodiments of the method can include: collecting a set of samples from a set of individuals comprising a set of first individuals who respond (e.g., positively; etc.) to one or more therapies (e.g., for one or more microbiome-related conditions; for any suitable conditions; etc.) but present side-effects (e.g., side-effect symptoms; etc.), and a set of second individuals who respond (e.g., positively; etc.) to the one or more therapies and do not present side-effects; determining one or more microbiome datasets based on the set of samples, wherein the microbiome datasets comprise at least one of: a microbiome taxonomic composition dataset, a microbiome function dataset, a microbiome composition diversity dataset, and a microbiome functional diversity dataset; extracting a set of microbiome features from the at least one dataset, wherein the set of microbiome features facilitate differentiation between responders with side-effects and responders without side-effects; determining a companion diagnostics model (e.g., classifier) based on the set of microbiome features; and/or generating and/or performing companion diagnostics using the companion diagnostics model, such as for differentiating responders with side-effects from responders without side-effects, in relation to the one or more therapies. However, any suitable individuals can be used for samples; any suitable microbiome data can be used for determining features; any suitable type of model can be generated for facilitating companion diagnostics; and companion diagnostics models can be used for any suitable purpose (e.g., taxonomic composition data such as a composition diversity dataset; functional dataset such as a functional diversity dataset; etc.).

Embodiments of the system and/or methods (e.g., companion diagnostics, using companion diagnostics, etc.) can be used for any suitable microorganism-related conditions (e.g., any suitable conditions for which the microbiome can affect drug efficacy and/or processing by a user; etc.).

Microorganism-related conditions (e.g., for which one or more severity metrics can be determined and/or applied; etc.) can include one or more of: diseases, symptoms, causes (e.g., triggers, etc.), disorders, associated risk (e.g., propensity scores, etc.), associated severity, behaviors (e.g., caffeine consumption, alcohol consumption, sugar consumption, habits, diets, etc.), and/or any other suitable aspects associated with microorganism-related conditions. Microorganism-related conditions can include one or more disease-related conditions, which can include any one or more of: gastrointestinal-related conditions (e.g., irritable bowel syndrome, inflammatory bowel disease, ulcerative colitis, celiac disease, Crohn's disease, bloating, hemorrhoidal disease, constipation, reflux, bloody stool, diarrhea, etc.); allergy-related conditions (e.g., allergies and/or intolerance associated with wheat, gluten, dairy, soy, peanut, shellfish, tree nut, egg, etc.); locomotor-related conditions (e.g., gout, rheumatoid arthritis, osteoarthritis, reactive arthritis, multiple sclerosis, Parkinson's disease, etc.); cancer-related conditions (e.g., lymphoma; leukemia; blastoma; germ cell tumor; carcinoma; sarcoma; breast cancer; prostate cancer; basal cell cancer; skin cancer; colon cancer; lung cancer; cancer conditions associated with any suitable physiological region; etc.); cardiovascular-related conditions (e.g., coronary heart disease, inflammatory heart disease, valvular heart disease, obesity, stroke, etc.); anemia conditions (e.g., thalassemia; sickle cell; pernicious; fanconi; haemolyitic; aplastic; iron deficiency; etc.); neurological-related conditions (e.g., ADHD, ADD, anxiety, Asperger's syndrome, autism, chronic fatigue syndrome, depression, etc.); autoimmune-related conditions (e.g., Sprue, AIDS, Sjogren's, Lupus, etc.); endocrine-related conditions (e.g., obesity, Graves' disease, Hashimoto's thyroiditis, metabolic disease, Type I diabetes, Type II diabetes, etc.); skin-related conditions (e.g., acne, dermatomyositis, eczema, rosacea, dry skin, psoriasis, dandruff, photosensitivity, rough skin, itching, flaking, scaling, peeling, fine lines or cracks, gray skin in individuals with dark skin, redness, deep cracks such as cracks that can bleed and lead to infections, itching and scaling of the skin in the scalp, oily skin such as irritated oily skin, skin sensitivity to products such as hair care products, imbalance in scalp microbiome, etc.); Lyme disease conditions; communication-related conditions; sleep-related conditions; metabolic-related conditions; weight-related conditions; pain-related conditions; genetic-related conditions; chronic disease; and/or any other suitable type of disease-related conditions. In variations, microorganism-related conditions can include one or more women's health-related conditions (e.g., reproductive system-related conditions; etc.).

In variations, microorganism-related conditions can include mosquito-related conditions, such as conditions including and/or associated with mosquito bites, malaria, and/or other suitable conditions associated with mosquitos. In variations, microorganism-related conditions can include insect-related conditions associated with any suitable insect bites and/or insects. In specific examples, microbiome-derived companion diagnostics can be used for evaluating therapies associated mosquito-related conditions.

Additionally or alternatively, microorganism-related conditions can include one or more human behavior conditions which can include any one or more of: diet-related conditions (e.g., caffeine consumption, alcohol consumption, sugar consumption, artificial sweetener consumption, omnivorous, vegetarian, vegan, sugar consumption, acid consumption other food item consumption, dietary supplement consumption, dietary behaviors, etc.), probiotic-related behaviors (e.g., consumption, avoidance, etc.), habituary behaviors (e.g., smoking; exercise conditions such as low, moderate, and/or extreme exercise conditions; etc.), menopause, other biological processes, social behavior, other behaviors, and/or any other suitable human behavior conditions. Conditions can be associated with any suitable phenotypes (e.g., phenotypes measurable for a human, animal, plant, fungi body, etc.). In variations, portions of embodiments of the method and/or system can be used for facilitating diagnostics and/or facilitating promoting (e.g., providing; recommending; etc.) of one or more targeted therapies to users suffering from one or more microorganism-related conditions (e.g., skin-related conditions, etc.), such as based on one or more severity metrics and/or other suitable data.

In variations, samples (e.g., described herein), microorganism-related conditions, companion diagnostics, and/or associated therapies can correspond to and/or be associated with one or more body sites including at least one of a gut body site (e.g., corresponding to a body site type of a gut site), a skin body site (e.g., corresponding to a body site type of a skin site), a nose body site (e.g., corresponding to a body site type of a nose site), a mouth body site (e.g., corresponding to a body site type of a mouth site), a genitals body site (e.g., corresponding to a body site type of a genital site) and/or any suitable body sites located at any suitable part of the body.

Companion diagnostics models and/or any suitable approaches for generating, determining, and/or using microbiome-derived companion diagnostics, and/or embodiments of the method and/or system, can include, apply, use, train, generate, update, and/or otherwise process any one or more of: supervised learning (e.g., using logistic regression, using back propagation neural networks, using random forests, decision trees, etc.), unsupervised learning (e.g., using an Apriori algorithm, using K-means clustering), semi-supervised learning, a deep learning algorithm (e.g., neural networks, a restricted Boltzmann machine, a deep belief network method, a convolutional neural network method, a recurrent neural network method, stacked auto-encoder method, etc.) reinforcement learning (e.g., using a Q-learning algorithm, using temporal difference learning), a regression algorithm (e.g., ordinary least squares, logistic regression, stepwise regression, multivariate adaptive regression splines, locally estimated scatterplot smoothing, etc.), an instance-based method (e.g., k-nearest neighbor, learning vector quantization, self-organizing map, etc.), a regularization method (e.g., ridge regression, least absolute shrinkage and selection operator, elastic net, etc.), a decision tree learning method (e.g., classification and regression tree, iterative dichotomiser 3, C4.5, chi-squared automatic interaction detection, decision stump, random forest, multivariate adaptive regression splines, gradient boosting machines, etc.), a Bayesian method (e.g., naïve Bayes, averaged one-dependence estimators, Bayesian belief network, etc.), a kernel method (e.g., a support vector machine, a radial basis function, a linear discriminate analysis, etc.), a clustering method (e.g., k-means clustering, expectation maximization, etc.), an associated rule learning algorithm (e.g., an Apriori algorithm, an Eclat algorithm, etc.), an artificial neural network model (e.g., a Perceptron method, a back-propagation method, a Hopfield network method, a self-organizing map method, a learning vector quantization method, etc.), an ensemble method (e.g., boosting, boostrapped aggregation, AdaBoost, stacked generalization, gradient boosting machine method, random forest method, etc.), and/or any suitable artificial intelligence approach.

Further embodiments include systems and methods for microbiome modulation.

Alkylation of DNA has been associated with development of cancer conditions and/or other conditions. Colibactin is a genotoxin strongly associated with cancer; however, its metabolism is not fully understood. Colibactin's potential association/similarity with DNA alkylate-type compounds may indicate that colibactin could covalently modify DNA, in addition or alternative to other similar metabolites produced by microorganisms from the microbiome (e.g., gut microbiome, microbiome from any suitable body site.

Embodiments of the system and/or method can include inhibiting for colibactin and/or analogous toxins that alkylate DNA, such as for improving one or more cancer conditions and/or suitable DNA alkylation-associated conditions (e.g., by inhibiting association with producing and/or developing one or more cancer conditions; etc.). As such, embodiments of the system can include one or more diagnostic systems for diagnosing cancer conditions and/or DNA alkylation-associated conditions based on colibactin (e.g., based on its association with cancer conditions; etc.); and/or one or more therapeutic compositions (e.g., gene modulation compositions, such as compositions operable to modulate one or more genes; etc.) for treating one or more cancer conditions and/or DNA alkylation-associated conditions by inhibiting colibactin and/or analogous toxins that alkylate.

Embodiments of the system and/or method can include detecting and/or identifying metabolites, from and/or derived from the microbiome (e.g., gut microbiome; microbiome associated with stool, genital, vaginal, nasal, mouth, skin and/or any suitable body sites or non-body sites; etc.), that are structurally and/or functionally similar to colibactin, such as with high potential to covalently modify DNA (e.g., alkylate DNA); and/or using one or more bioinformatics approaches (e.g., including machine learning and/or AI techniques, etc.) to identify and/or improve specific inhibitors that can block the action of colibactin and/or colibactin-like metabolites, and its use for therapeutic compositions (e.g., treatments, etc.) against cancer disease and/or abnormal cell growth associated condition.

Additionally or alternatively, embodiments of the system and/or method can include and/or function to provide diagnostics for cancer conditions and/or cancer related conditions (e.g. colorectal cancer disease) by using colibactin and/or colibactin-type molecules for diagnostics, and/or provide a therapy based on specific colibactin and/or colibactin-like metabolites.

Embodiments of the system and/or method can include any suitable gene modulation (e.g., gene editing) and/or gene depletion techniques (e.g. point mutation, CRISPR technology, CRISPR-Cas9, etc.), such as in order to delete and/or block transcription of gene related with producing colibactin, colibactin-like molecule and/or any precursor gene, in order to inhibit progression of cancer disease or cancer related condition, as a complete or support therapy. Additionally or alternatively, gene modulation and/or gene depletion techniques can include any one or more of: induced mutations, CRISPR, CRISPR-Cas9, gene knockout, gene knockin, mutagenesis (e.g., directed, site-directed, PCR mutagenesis, insertional, transposon mutagenesis, signature tagged, sequence saturation, etc.), and/or any suitable techniques. Endonucleases usable with CRISPR can include any one or more of: Cas9, Cpf1, SauCas9, or any other CRISPR type endonuclease that targets DNA or RNA.

In an embodiment, the method and/or system can include identifying colibactin-like molecules from different sources (e.g., biological sources, non-biological sources, chemical sources, by synthesis, etc.) in order to use it for determination (e.g., elaboration, etc.) of inhibitors, improvement of current inhibitors, and/or modification of a non-inhibitor molecule (e.g. small molecule, peptides, synthetic peptides, short chain fatty acids, etc.) into an inhibitor molecule, for diagnostic and/or therapeutic use.

In an embodiment, the method and/or system can include the use of colibactin and/or colibactin-like genes as a biomarker for diagnostics and/or therapy, based on detection of colibactin and/or colibactin gene in pathogenicity island of several microorganisms within microbiome. Alternatively or additionally, embodiments can include the use of bacteriophages for detection, mutation and/or any other suitable method to inhibit colibactin and/or colibactin-like gene for specific microorganisms from microbiome.

Additionally or alternatively, any suitable approaches described herein (e.g., gene depletion approaches) can be applied for modulating (e.g., removing) any suitable microbial functions (e.g., in addition to or alternative to colibactin-related functionality, etc.).

Embodiments of the method and/or system can include, apply, process, and/or otherwise use microbiome samples (e.g., from different body sites) and/or can be applied to modulating microbiomes from any suitable body sites, and/or can be used directly (e.g., therapeutically; for diagnostics; etc.) in relation to any suitable body sites. Body sites can include any one or more of stool, gut, genital, vaginal, nasal, mouth, skin and/or any suitable types of body sites. Embodiments of the method and/or system can include, apply, process, and/or otherwise be used for treating the host in vivo, samples ex vivo and/or in vitro, fecal samples for transplant, and/or any other sample, and/or using CRISPR-Cas (and/or other suitable CRISPR-associated mechanisms; and/or any suitable gene modulation and/or gene depletion mechanisms; etc.) to target colibactin-related genes (e.g., genes resulting in colibactin production; genes resulting in production of molecules with functionality analogous to colibactin; etc.), and/or any suitable genes associated with bacteria, fungi, parasites, and/or any other microorganisms (e.g., for modifying any suitable microbial functions; etc.). Delivery systems (e.g., of compositions described herein; of any suitable components described herein; etc.) can include one or more of: transformation, conjugation, natural or engineered phages, microinjection, electroporation, hydrodynamic injection, viral vectors, nucleofection, membrane deformation, nanoparticles, zeolitic imidazole frameworks, biotinylated oligonucleotides coupled or not with RNA or DNA aptamers, polymers, lipids, DNA nanoclews, zwitterionic amino-lipid nanoparticles, liposomes, the use of probiotics or genetically engineered microorganism carrying CRISPR/Cas systems that can be transferred to the target organisms from the microbiome, an/or any suitable delivery mechanisms.

### Example 1: Method and System for Vaginal Panel Characterizations

In embodiments, a Clinical Vaginal Microbiome Panel, referred to herein as SmartJane v3, can be designed for detecting bacterial genera and/or species, and/or other microorganisms (e.g., fungi, protozoans, etc.) from any suitable microorganism taxa, from one or more human vaginal microbiome samples (and/or other suitable types of samples can be used from any suitable body sites, etc.) using culture-independent sequencing. In embodiments, the test can use precision sequencing, a combination of amplicon sequencing (e.g. using primers targeting the variable 4 (V4) region of the 16S rRNA genes) with full metagenomic sequencing. In examples, libraries can then be sequenced using a next generation sequencing platform and/or other suitable platform. Additionally or alternatively, any suitable portions of embodiments described herein can include, apply, and/or be associated with high-throughput sequencing (e.g., facilitated through high-throughput sequencing technologies; massively parallel signature sequencing, Polony sequencing, 454 pyrosequencing, Illumina sequencing, SOLiD sequencing, Ion Torrent semiconductor sequencing, DNA nanoball sequencing, Heliscope single molecule sequencing, Single molecule real time (SMRT) sequencing, Nanopore DNA sequencing, etc.), any generation number of sequencing technologies (e.g., second-generation sequencing technologies, third-generation sequencing technologies, fourth-generation sequencing technologies, etc.), amplicon-associated sequencing (e.g., targeted amplicon sequencing), metagenome-associated sequencing (e.g., metatranscriptomic sequencing, metagenomic sequencing, etc.), sequencing-by-synthesis, tunnelling currents sequencing, sequencing by hybridization, mass spectrometry sequencing, microscopy-based techniques, capillary sequencing, Sanger sequencing (e.g., microfluidic Sanger sequencing, etc.), pyrosequencing, nanopore sequencing (Oxford nanopore sequencing, and/or any suitable sequencing technologies.

In embodiments, a bioinformatics pipeline can use as input sequencing data to infer the bacterial genera and species (and/or other suitable microorganism taxa) present, and/or also consider microbial diversity analysis, and/or suitable microbiome composition and/or microbiome function analysis. Analyses (e.g., recommendations, evaluations, etc.) related to probiotics, prebiotics, and/or other suitable consumables and/or therapies can be included, determined, promoted, and/or provided, such as using the approaches described herein.

In embodiments, for this assay, the 16S V4 pipeline can include similar or same processes up to library quantification, as metagenomics will follow a completely independent process after DNA extraction. In embodiments, after separate quantification, 16S and metagenomics libraries will be combined and sequenced together in high output cassettes.

In examples, the following targets are included in v3.0:

Yeast genera: Candida

Yeast species: Candida albicans, Candida glabrata, Candida parapsilosis, Candida tropicalis, Candida krusei

Bacterial genera: Anaerococcus, Anaeroglobus, Arcanobacterium, Bulleidia, Eggerthella, Dialister, Finegoldia, Fusobacterium, Mobiluncus, Moryella, Leptotrichia, Veillonella

Bacterial species: Mycoplasma hominis

Protozoans: Trichomonas vaginalis

Therefore, in examples, SmartJane v3.0 considers the analytic validation of at least 10 bacterial targets that are detected by the 16S pipeline, and/or at least 6 yeast targets and/or at least 1 protozoan target that are detected by metagenomics. Additionally or alternatively, embodiments can include, analyze, and/or characterize any suitable number and/or type of targets (e.g., of any suitable microorganism taxa, etc.) using any suitable pipelines (e.g., 16S pipeline and/or metagenomics, etc.)

### Specific Examples of Analysis for 16S rRNA Sequences

Using a 16S sequences database (e.g. version 123 of the SILVA database) we determined theoretical (in silico) performance metrics for the taxonomic annotation of 16S V4 amplicons for all 32 taxa targeted by the assay.

To generate the taxonomic database used to implement the clinical bioinformatics pipeline described here, we first predicted the amplicons that would be produced by V4 primers for all the sequences in the database. The primers used were GTGCCAGCMGCCGCGGTAA (SEQ ID NO: 1) (forward) and GGACTACHVGGGTWTCTAAT (SEQ ID NO: 2) (reverse), where M is A or C, H is A, C or T, V is A, C or G and W is A or T. We allowed annealing with up to 2 mismatches. The resulting predicted amplicons were subsequently inspected for degenerate bases. Degenerate amplicons that expand to more than 20 possible non-degenerate sequences were regarded as bad quality sequences and were eliminated from the database, whereas those that expanded to less than 20 possible sequences were kept expanded as each of their non-degenerate sequences. The amplicons were further processed using pair-end sequencing, so that they were represented by a forward read containing the forward primer and 125 bp to the 3′ end of the forward primer, and a reverse read containing the reverse primer and 124 bp to the 3′ end of the reverse primer. Finally, primers were removed, and the remainder of the reads (125 bp after the forward primer plus 124 bp after the reverse primer) were concatenated and stored in an amplicon database.

### Examples of Analysis for Metagenomics Sequences

Metagenomics is a pipeline that captures all the DNA present in a sample. In that context and given the nature of the metagenomics pipeline theoretical (in silico) performance metrics were determined for the taxonomic classification based in k-mers obtained using the bioinformatics pipeline. To generate the taxonomic database used to implement the clinical metagenomics bioinformatics pipeline the NT database was first curated. To do this NT sequences were filtered if they come from a list of selected ids that include: Bacteria, Archaea, Viruses, Fungi, micro-eukarya and/or Human and/or any suitable microorganism taxa.

The metagenomics pipeline generates random fragments of DNA ranged between 200 and 600 pb approximately. Different reads that can or cannot be overlapped were produced because of the use of pair-end sequencing. Moreover, these DNA regions could be non-informative. This means that a specific region can be shared by multiple organisms. Thus, taxonomy classification is preferably based on sequence similarity of k-mers originated from pair-end reads using 100% identity over 100% of the length against k-mers of sequences in NT curated databases; however, any suitable similarity (e.g., any suitable identity percentage; over any suitable length; etc.) conditions and/or any suitable criteria can be used. The k-mers present in a curated database for each species or genus are what define the elements of the confusion matrices and therefore the performance metrics for predictions.

However, any suitable approaches described herein can include, be applied with, can correspond to, and/or can be otherwise associated with approaches in and/or analogous to U.S. application Ser. No. 16/115,542 filed 28 Aug. 2018, which is herein incorporated in its entirety by this reference.

### Example 2: Method and System for Diagnostics and/or Treatment for Cancer Related Conditions Associated with HPV Infection

Embodiments of the system (e.g., therapeutic compositions; etc.) and/or method can include detection, identification, determination, generation, and/or promotion of (e.g., provision; administration; etc.) of one or more therapies (e.g., therapeutic composition; etc.) for treatment of HPV infection, lesions and progression and/or HPV-associated cancer conditions and/or related conditions.

Embodiments of the method and/or system can detection, diagnose, identify and/or otherwise characterize one or more HPV-associated conditions (e.g., HPV infection, any suitable HPV strains, HPV-related symptoms, etc.) through including, implementing, and/or otherwise applying any suitable approaches described in and/or analogous to that described in U.S. application Ser. No. 16/115,542 filed 28 Aug. 2018, and U.S. application Ser. No. 15/198,818 filed 30 Jun. 2016, which are each herein incorporated in their entirety by this reference; and promote (e.g., provide, generate, administer, etc.) one or more specific treatments based on aminolevulinic acid (ALA) and/or equivalent(s) (e.g., delta aminolevulinic acid, etc.) in the affected region and/or specific and/or related region(s) (e.g., cervix, etc.) that after a set of time (e.g., 4 hrs., suitable periods of time, etc.) is transformed into a fluorescent compound (e.g., protoporphyrin IX) and accumulates in lesions; after that, treatment involves continuing with irradiating the corresponding zone (e.g., region) with accumulation of fluorescent transformed compound, producing reactive oxygen species that destroy DNA in the infected cells. In a specific example, a repeated session is required for a defined period of time (e.g., 48 hrs, etc.) in order to ensure results and success of treatment.

Embodiments of the method and/or system can include diagnostics for one or more HPV-related conditions (e.g., HPV strains, etc.), in order to specify zone (e.g., location, body site, severity, etc.) of infection and/or specific strains; and/or therapeutics (e.g., based on diagnostic characterization; etc.) with a corresponding two-session-treatment based on aminolevulinic acid and/or analogous and/or improved compounds able to accumulate on the lesion, marked zone, and transform by any suitable approach into a reactive oxygen species, such as for destroying tumors by destroying selectively or mostly specifically infected cells. In a specific example, the first session, should be at least repeated within at least 48 hrs, in order to provide an effective treatment.

Embodiments of the method and/or system can additionally or alternatively include improvement of the best candidates for treatment through use of molecules, according to type of infection regarding different HPV strains.

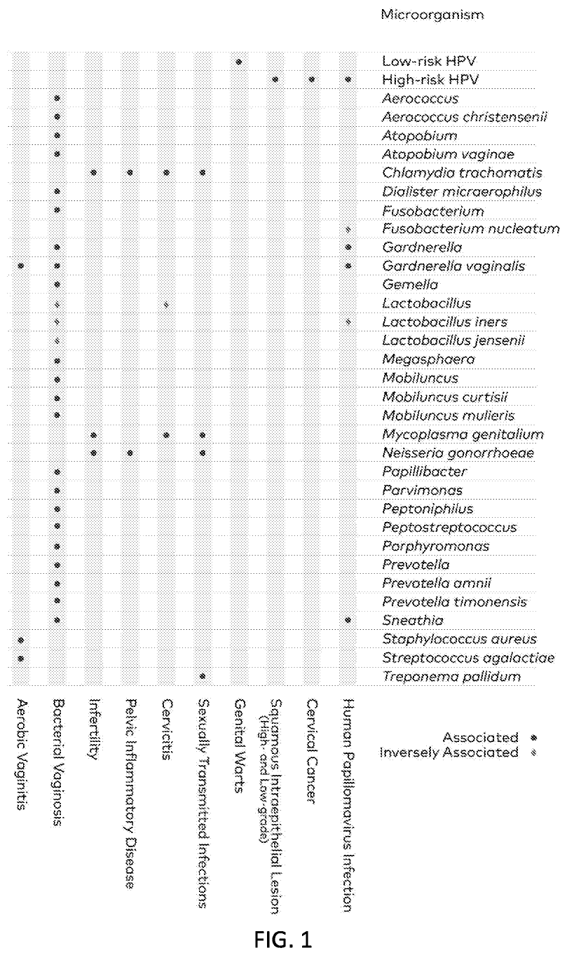

Based on scientific literature, 32 bacterial targets with clinical relevance for women's reproductive tract health were selected (FIG. 1), including Lactobacillus, Sneathia, Gardnerella, and 4 pathogens involved in STI (Chlamydia trachomatis, Mycoplasma genitalium, Neisseria gonorrhoeae, and Treponema pallidum). In addition, the assay targets 14 hrHPV (16, 18, 31, 33, 35, 39, 45, 51, 52, 56, 58, 59, 66, 68) and 5 lrHPV types (low-risk types associated with genital warts, i.e.: 6, 11, 42, 43, 44).

FIG. 1 shows a matrix of 32 bacterial targets and 19 HPV targets covered by the women's health assay and their associated health conditions. Dots indicate targets positively associated with the conditions, while diamonds indicate inverse associations.

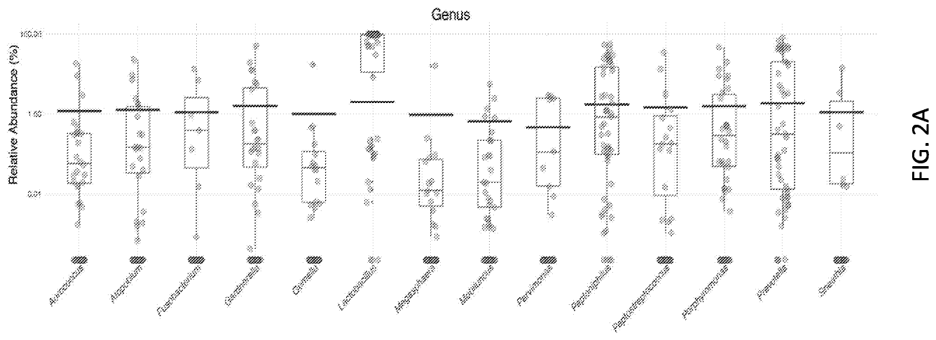

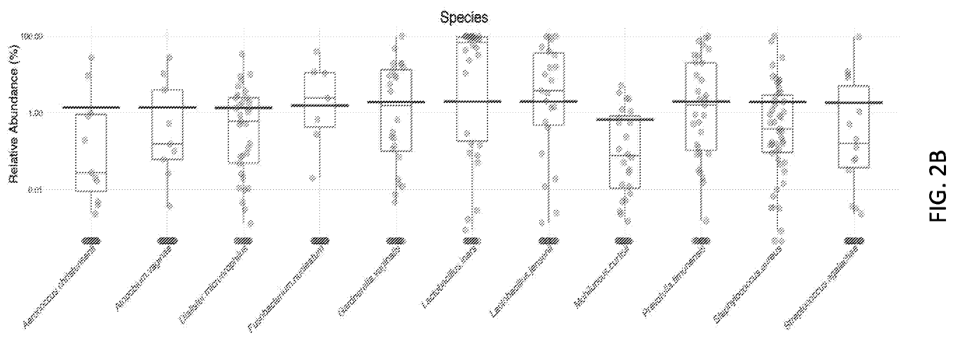

All targets were selected based on in silico performance in sequences from online databases. FIGS. 2A and 2B show healthy ranges for the bacterial targets in the assay according to an embodiment of the present disclosure. A set of 50 vaginal samples, each from a different woman, was selected based on the self-reported answers given to survey questions indicating general and vaginal health. Not all of the taxa used in the assay were plotted, as some had no abundance values for this healthy cohort. No HPV reads were found in this cohort.

The performance of the targets (detection in vaginal samples, limit-of-detection in diluted pools) was assessed using synthetic DNA fragments (not shown). The performance of the hr- and lrHPV genotyping portion of the assay was evaluated against the digene HPV HC2 assay.

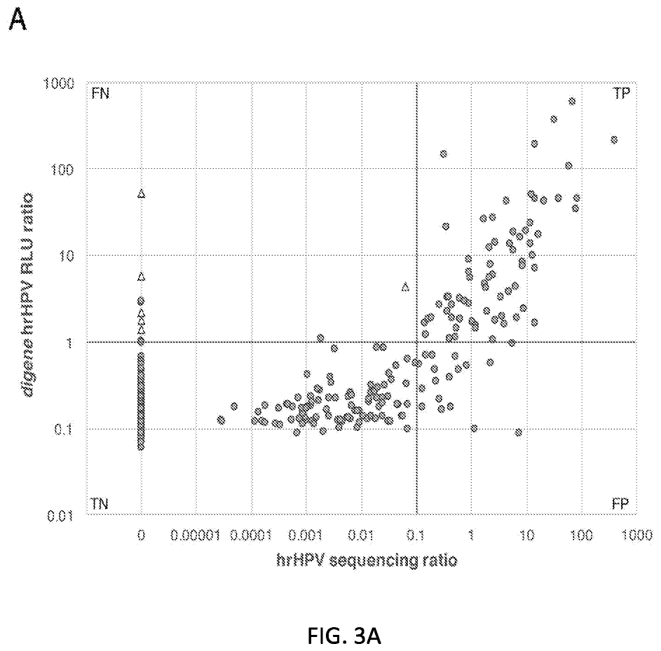

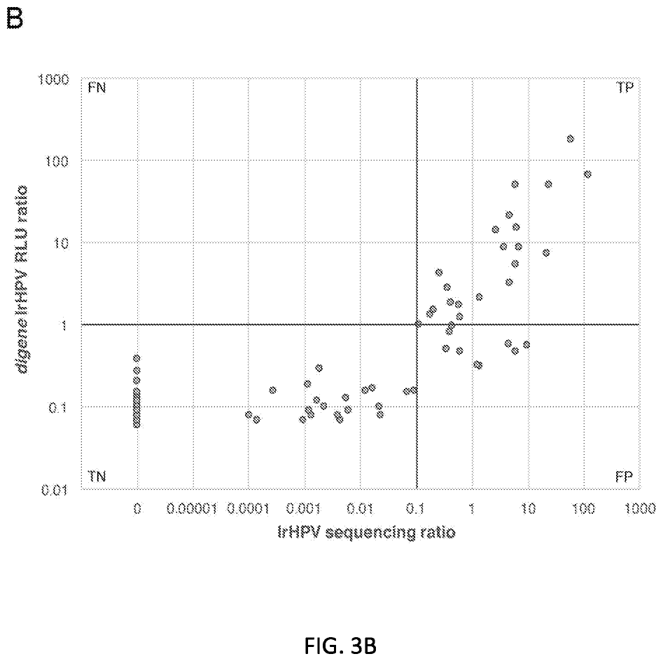

FIGS. 3A and 3B show a comparison of hrHPV and lrHPV amplification and genotyping to the digene HC2 HPV assay in accordance with an embodiment of the present disclosure. DNA from 718 vaginal samples was extracted and tested by PCR amplification and sequencing using HPV primers, and additionally used directly in the digene assay using the HC2 hrHPV (panel A; 601 samples) or lrHPV (panel B; 148 samples) probe mix. The open triangles in A depict samples positive in the hrHPV digene assay that contained lrHPV reads only.

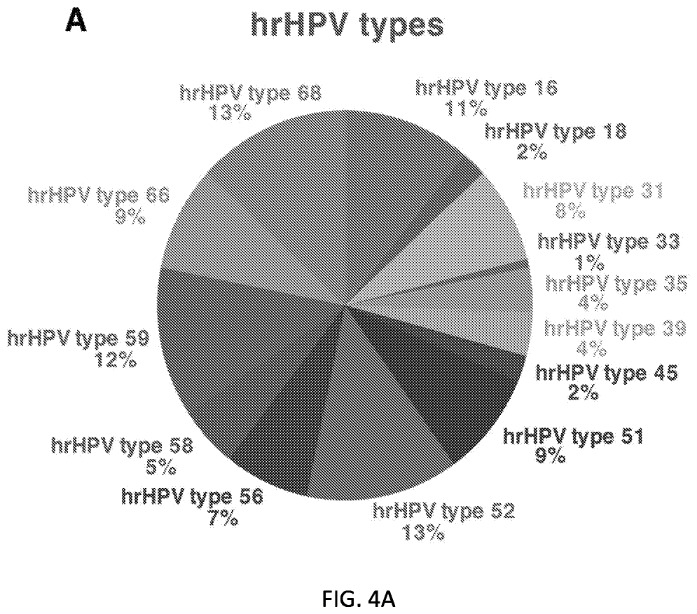

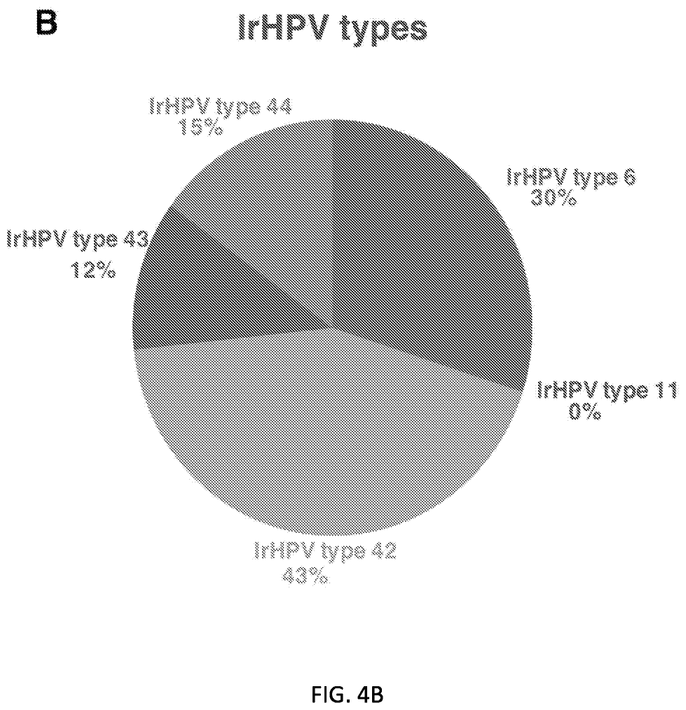

FIGS. 4A and 4B show a distribution of the 19 HPV types in one of the studies. Of these, 142 samples were positive for at least one of the 19 HPV types (14 hrHPV, panel A; 5 lrHPV, panel B) validated in this study. In 35 of these, two or more HPV types were found.

However, any suitable approaches described herein can include, be applied with, can correspond to, and/or can be otherwise associated with approaches in and/or analogous to U.S. application Ser. No. 16/115,542 filed 28 Aug. 2018, and U.S. application Ser. No. 15/198,818 filed 30 Jun. 2016 (which are each herein incorporated in their entirety by this reference), such as for characterizing one or more HPV-related conditions (e.g., HPV infection by any suitable HPV strains), and where therapeutic approaches described herein can be correspondingly promoted (e.g., promoted based on, in response to, subsequent to, and/or in any suitable time, frequency, and/or fashion in relation to a characterization of one or more female reproductive system-related characterizations; etc.).

The methods and/or system of the embodiments can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a patient computer or mobile device, or any suitable combination thereof. Other systems and methods of the embodiments can be embodied and/or implemented at least in part as a machine configured to receive a computer-readable medium storing computer-readable instructions. The instructions can be executed by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor, though any suitable dedicated hardware device can (alternatively or additionally) execute the instructions.

The FIGURES of U.S. application Ser. No. 16/115,542 (which is incorporated herein in its entirety) illustrate the architecture, functionality and operation of possible implementations of systems, methods and computer program products according to preferred embodiments, example configurations, and variations thereof. In this regard, each block in the flowchart or block diagrams may represent a module, segment, step, or portion of code, which comprises one or more executable instructions for implementing the specified logical function(s). It should also be noted that, in some alternative implementations, the functions noted in the block can occur out of the order noted in the FIGURES of U.S. application Ser. No. 16/115,542. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

As a person skilled in the art will recognize from the previous detailed description and from the figures and claims, modifications and changes can be made to the embodiments of the invention without departing from the scope of this invention as defined in the following claims.

What is claimed is:

1. A method of treating a cancer related condition associated with HPV infection, the method comprising:
generating a disease characterization model generated by analyzing one or more microbiome features obtained from microbiome samples from a population having the HPV-associated condition;
collecting a microbiome sample from an individual;
generating one or more microbiome datasets based on the microbiome sample from the individual and extracting a set of microbiome features from the one or more microbiome datasets;
determining whether the individual has the HPV-associated condition based on the set of microbiome features and the disease characterization model; and
upon a diagnosis that the individual has the HPV-associated condition, administering a treatment based on aminolevulinic acid (ALA) in a region affected by the HPV-associated condition.

2. The method of claim 1, wherein administering the treatment based on ALA comprises irradiating the region after a set time during which the ALA or the analog thereof transforms into a fluorescent compound, the irradiating being characterized by production of reactive oxygen species in the irradiated region.

3. The method of claim 1, further comprising characterizing the HPV-associated condition following the administration of the treatment based on ALA to determine whether a follow-on ALA based treatment is required.

4. The method of claim 3, wherein upon a determination that a follow-on ALA based treatment is required, administering a second dose of ALA or an analog thereof in the region after a predefined period and irradiating the region after a set time during which the ALA or the analog thereof transforms into a fluorescent compound, the irradiating being characterized by production of reactive oxygen species in the irradiated region.

5. The method of claim 1, wherein identifying the HPV-associated condition comprises analyzing predetermined bacterial targets and HPV strains in the microbiome sample from the individual.

6. The method of claim 5, wherein the HPV strains are selected from the group consisting of 14 hrHPV and 5 lrHPV strains.

7. A method of treating a DNA alkylation-associated condition, the method comprising:
obtaining a microbiome sample from an individual;
detecting a DNA alkylation-associated condition based on presence and/or amount of one of colibactin or colibactin-like compound in the microbiome sample; and
administering a colibactin-inhibiting treatment to the individual for treating the DNA alkylation-associated condition.

8. The method of claim 7, wherein the treatment comprises administering a colibactin-inhibiting compound to the individual.

9. The method of claim 7, wherein the treatment comprises administering a gene modulation therapy to the individual, the gene modulation therapy configured to delete a gene related with producing colibactin or block transcription of a gene related with producing colibactin.

10. The method of claim 9, wherein the gene modulation therapy is selected from the group consisting of: induced mutations, CRISPR, CRISPR-Cas9, gene knockout, gene knockin, mutagenesis.

11. The method of claim 7, wherein the microbiome sample from the individual is obtained from a body site selected from the group consisting of: stool, gut, genital, vaginal, nasal, mouth, and skin.

12. The method of claim 7, wherein the DNA alkylation-associated condition comprises a cancer disease or an abnormal cell growth associated condition.

13. A method for characterizing compatibility of a drug for a user, the method comprising:
collecting a set of microbiome samples from a set of individuals comprising a set of first individuals who respond to a therapy for a microbiome-related condition and a set of second individuals who do not respond to the therapy for the microbiome-related condition;
determining one or more microbiome datasets based on the set of microbiome samples;
extracting a set of microbiome features from the one or more microbiome datasets, the microbiome features facilitating differentiation between individuals who respond and individuals who do not respond to the therapy;
determining a companion diagnostics model based on the set of microbiome features; and
determining the compatibility of the drug for the user using the companion diagnostics model.

14. The method of claim 13, wherein the microbiome datasets comprise at least one of: a microbiome taxonomic composition dataset, a microbiome function dataset, a microbiome composition diversity dataset, and a microbiome functional diversity dataset.

15. The method of claim 13, wherein the companion diagnostics model provides a criterion for differentiating responders from non-responders in relation to the therapy.

16. The method of claim 13, wherein the therapy comprises administering a drug, and the companion diagnostics model provides a criterion indicating at least one of: a type of drug, dosage for the drug, risk factors associated with the drug, pharmacogenetics, an expected response to the drug, side effects associated with the drug, and toxicity associated with the drug.

17. The method of claim 13, wherein the microbiome-related condition includes one or more selected from the group consisting of: gastrointestinal-related conditions; allergy-related conditions; locomotor-related conditions; cancer-related conditions; cardiovascular-related conditions; anemia conditions; neurological-related conditions; autoimmune-related conditions; endocrine-related conditions; skin-related conditions; Lyme disease conditions; communication-related conditions; sleep-related conditions; metabolic-related conditions; weight-related conditions; pain-related conditions; genetic-related conditions; chronic disease; and one or more women's health-related conditions.

18. The method of claim 13, wherein the first individuals comprises a set of third individuals who respond positively to the therapy, but with side-effects, and a set of fourth individuals who respond positively to the therapy without side-effects.


SEQUENCE LISTING (2 sequences)

SEQ ID NO: 1 - DNA, 19 bp, Artificial Sequence
  Synthetic sequence_primer (forward)
gtgccagcmgccgcggtaa

SEQ ID NO: 2 - DNA, 20 bp, Artificial Sequence
  Synthetic sequence_primer (reverse)
ggactachvgggtwtctaat